(19) United States
(12) Patent Application Publication
(10) Pub. No.: US 2005/0137166 A1
(43) Pub. Date: Jun. 23, 2005

(54) Use of cooling agents to relieve mild ocular irritation and enhance comfort

(75) Inventors: Bahram Asgharian, Arlington, TX (US); David L. Meadows, Colleyville, TX (US)

Correspondence Address: ALCON RESEARCH, LTD., R&D COUNSEL, Q-148, 6201 SOUTH FREEWAY, FORT WORTH, TX, 76134-2099, US

(73) Assignee: Alcon, Inc.

(21) Appl. No.: 11/000,729

(22) Filed: Dec. 1, 2004

Related U.S. Application Data
(60) Provisional application No. 60/531,499, filed on Dec. 19, 2003.

(51) Int. Cl. A61K 31/736; A61K 31/215; A61K 31/17; A61K 31/16; A61K 31/18

(52) U.S. Cl. 514/54; 514/616; 514/529; 514/602; 514/588; 514/472

(57) ABSTRACT

Ophthalmic compositions containing very low concentrations (e.g., 1 to 50 ppm) of cooling agents are described. The cooling agents are less volatile and less prone to causing ocular discomfort than agents previously utilized to obtain an ocular cooling effect, such as menthol. The cooling agents are preferably contained in a vehicle that forms a gel or partial gel upon application to the eye.

### CLAIM FOR PRIORITY

This application claims priority from U.S. Patent Application Ser. No. 60/531,499, filed Dec. 19, 2003.

### BACKGROUND OF THE INVENTION

The present invention is directed to the use of cooling agents to relieve mild ocular irritation and enhance ocular comfort. The invention is particularly directed to use of cooling agents in ophthalmic compositions utilized to relieve ocular redness, dry eye conditions and ocular allergies.

The use of cooling agents such as menthol to provide a cooling effect on the skin and in the oral cavity is known. Cooling agents have also been added to food products such as chewing gum or mints, as well as to cigarettes, in order to provide a sensation of “coolness or freshness” during consumption. Menthol has also been added to topical pharmaceutical compositions to alleviate the sensation of inflammation and itch associated with bug bites and mild abrasions.

The sensation of coolness on the skin and mucosal surfaces resulting from the application of menthol is believed to be due to a specific action on sensory nerve endings. It is believed that cooling agents such as menthol exert their effect on cold receptors by interfering with the mobility of calcium ions across the cell membrane. Menthol has a mint order and it is quite volatile. It has been perceived as being irritating to the eye, and consequently has not been utilized extensively in ophthalmic preparations.

There have been prior attempts to provide ophthalmic compositions with a refreshing effect, for example, an eye drop marketed by the Japanese company, Rhoto, under the name “RhotoZi” has recently been introduced. This product contains camphor to provide a sensation of refreshment. Camphor is not, however, a cooling agent.

Japanese Patent Application No. 09-143064 (Taisho Pharmaceutical Co., Ltd.) describes the use of solutions containing menthol to treat eye fatigue.

Japanese Patent Application No. 09-132526 (Lion Corp.) describes ophthalmic solutions containing menthol, camphor and/or borneol. The solutions are said to provide a cooling effect when applied to the eyes.

The following publications may be referred to for further background regarding prior attempts to treat dry eye or eye fatigue attributed to the operation of computer display terminals:
U.S. Pat. No. 5,830,913 (Senju Pharmaceutical Co., Ltd.; 12-sulphode-hydroabietic acid);
Japanese Patent Application No. 08-133967 (Eisai Co., Ltd.; teprenone); and
French Patent No. 2 656 529 (Société Civille Anben; cytidine).

### SUMMARY OF THE INVENTION

The present invention is directed to the provision of ophthalmic compositions that produce a cooling effect or sensation when topically applied to the eyes of humans or other mammals.

The present invention is based on the surprising finding that the use of very low concentrations of cooling agents (i.e., a few parts per million) provides a refreshing cooling effect without causing ocular discomfort. Furthermore, it has been found that there is a dose dependency for the cooling intensity and duration of cooling sensation.

The use of cooling agents that are capable of providing an ocular cooling effect at very low concentrations has several advantages.

The use of very low concentrations of cooling agents reduces the risk of producing an anesthetic effect when the agents are applied to the eye. The avoidance of an anesthetic effect is important, because this effect can mask ocular infections, injuries, or other conditions that require immediate medical attention.

The ability to produce a cooling effect with a very low concentration of cooling agent also reduces the risk of ocular irritation. The avoidance of ocular irritation or discomfort is important, particularly in patients whose eyes are already irritated due to dry eye symptoms, ocular fatigue, or other conditions.

Finally, the use of an excessive amount of cooling agent to achieve a cooling effect can actually produce a heating effect; the ability to produce a cooling effect with a very low concentration of a highly potent agent also helps to avoid this problem.

The cooling agents utilized in the compositions of the present invention are non-volatile and produce very little, if any, odor when the compositions are applied to the eye. In contrast, menthol is volatile, has a mint odor, and is generally perceived to be irritating when applied to the eyes.

The cooling agents utilized in the present invention are preferably contained in compositions that form a gel or partial gel upon application to the eye. The use of such compositions to apply the cooling agents to the eye further enhances the effectiveness of the agents, thereby facilitating the use of very low concentrations of the agents.

Thus, the present invention provides improved compositions and methods for providing an ocular cooling effect in patients that are experiencing ocular irritation or discomfort associated with dry eye conditions, ocular allergic symptoms, ocular fatigue, or other ophthalmic conditions.

### DETAILED DESCRIPTION OF THE INVENTION

The compounds utilized in the ophthalmic compositions of the present invention to produce a “cooling” effect span a range of different chemical classes, such as menthyl esters, carboxamides, menthane glycerol ketals, alkyl substituted ureas, sulfonamides, terpene analogs, furanones, and phosphine oxides. The compounds must be non-irritating, non-volatile, and stable in aqueous solutions at physiological pH and osmolality conditions.

N,2,3-Trimethyl-2-isopropylbutamide, commercially available as “WS-23” from Millennium Chemicals, is one of the preferred cooling agents of the present invention. It is most effective at a concentration of 10 to 20 ppm.

Among terpene analogs, Cyclohexanecarboxamide, N-ethyl-5-methyl-2-(1-methylethyl), is the preferred compound. It is commercially available from Mellenium Chemicals under the trade name “WS-3”. The optimal concentration is from 1 to 5 PPM.

Both of the above-identified cooling agents have FDA GRAS and EU registration for flavoring substances.

Menthol derivatives that may be utilized include menthone glycerin acetyal and menthyl lactate, which are commercially available from Haramann & Reimer (H&R) under the trade name of “Frescolat”. The above-cited cooling agents are approved in the European Union for use as flavoring agents.

(−)-Menthoxypropane-1,2-diol and (−)-Isopulegol, which are sold under trade names “Coolant agent 10” and “Coolant agent P” by Takasago International, may also be utilized in the ophthalmic compositions of the present invention.

Relative to furanones, 4-methyl-3-(1-pyrrolididinyl)-2[5H]-furanone is the most preferred compound. It is thirty-five times more powerful in the mouth and over 500 times more powerful on the skin than menthol. Consequently, it is possible to use a very low concentration of this compound to provide an ocular cooling effect.

The compositions of the present invention will contain one or more of the above-described cooling agents in an amount sufficient to produce a cooling effect or sensation when a small amount (e.g., one or two drops) of a composition containing the agent(s) is topically applied to the eye. The amount of cooling agent required for this purpose is referred to herein as a “cooling effective amount” or “an amount sufficient to produce a cooling effect”.

The amount of cooling agent required for each composition will vary depending on the particular agent selected, the type of composition in which the agent is to be utilized (e.g., solution or gel), and other factors apparent to those skilled in the art. The compositions of the present invention will generally contain one or more of the cooling agents at a concentration of 1 to 50 parts per million (“ppm”). The optimal concentration ranges for certain preferred cooling agents have already been specified above.

The above-described cooling agents may be applied to the eye via various types of ophthalmically acceptable vehicles. The preferred type of vehicle is an aqueous solution containing a mixture of electrolytes and other components that simulate the content of human tears. Such vehicles are referred to herein as “artificial tear compositions”. Such compositions are described in U.S. Pat. No. 5,403,598 (Beck, et al.), the entire contents of which are hereby incorporated in the present specification by reference.

The cooling agents may also be used in a variety of other types of ophthalmic compositions, including compositions that contain cellulose derivatives (e.g., HPMC) or other agents that provide a lubricant or cushioning effect when applied to the eye, such as the galactomannan (e.g., hydroxypropyl guar) formulations described in U.S. Pat. No. 6,583,125 (Asgharian), the entire contents of which are hereby incorporated in the present specification by reference. The compositions described in the '125 patent form a gel or partial gel upon application to the eye. The use of such gel-forming compositions as vehicles for applying the above-described cooling gents to the eye is particularly preferred.

The compositions of the present invention may also contain one or more of active drug for treatment of ocular conditions such as dry eye, ocular allergy, inflammation, anti-infective, etc.

The compositions of the present invention may also be formulated so as to be compatible with contact lenses, particularly compositions that are applied to the eye while the lenses are being worn, so as to improve the comfort of the lenses on the eye (i.e., “comfort drop” compositions).

The compositions of the present invention may also contain a wide variety of other ingredients, such as tonicity-adjusting agents (e.g., sodium chloride or mannitol), surfactants (e.g., anionic surfactants, such as RLM 100, and nonionic surfactants, such as the poloxamines sold under the name “Tetronic®” and the poloxamers sold under the name “Pluronic®”), and viscosity adjusting agents. The present invention is not limited with respect to the types of ophthalmic compositions in which the cooling agents described herein are utilized.

The ophthalmic compositions of the present invention will be formulated so as to be compatible with the eye and/or contact lenses to be treated with the compositions. The ophthalmic compositions intended for direct application to the eye will be formulated so as to have a pH and tonicity which are compatible with the eye. This will normally require a buffer to maintain the pH of the composition at or near physiologic pH (i.e., 7.4) and may require a tonicity agent to bring the osmolality of the composition to a level at or near 210-320 milliosmoles per kilogram (mOsm/kg). The formulation of compositions for disinfecting and/or cleaning contact lenses will involve similar considerations, as well as considerations relating to the physical effect of the compositions on contact lens materials and the potential for binding or absorption of the components of the composition by the lens. The compositions will generally be formulated as sterile aqueous solutions.

### EXAMPLE 1

The following formulation further illustrates the compositions of the present invention:

| | CONCENTRATION |
|---|---|
| COMPONENT | (w/v %) |
| Polyethylene Glycol 400 | 0.40 |
| Propylene Glycol | 0.30 |
| Hydroxypropyl guar | 0.18 |
| Boric Acid | 1.0 |
| Sodium Chloride | 0.10 |
| Potassium Chloride | 0.13 |
| Calcium Chloride | 0.0053 |
| Magnesium Chloride | 0.0064 |
| Zinc Chloride | 0.00015 |
| Polyquaternium-1 | 0.001 |
| Cooling Agent | 1-50 ppm |
| Purified Water | QS 100 w/v |

The above composition is prepared in 2 parts. In part 1, hydroxypropyl guar is dispersed in a portion of purified water and allowed to hydrate. The solution is then polish filtered and sterilized. In part 2, the remaining of the ingredients are dissolved in remaining portion of purified water and allowed to dissolve. The solution is then sterile filtered using a 0.2 μm membrane filter. Part 1 and 2 solutions are combined aseptically and pH of the solution is adjusted to target pH of 7.0 with NaOH.

### EXAMPLE 2

Compositions containing the cooling agents WS-3 and WS-23 in the vehicle described in Example 1 were prepared. The compositions were evaluated relative to the ability to produce a cooling sensation when topically applied to the eyes of human patients. The compositions were also evaluated with respect to the creation of ocular discomfort.

A series of compositions, which contained the cooling agent WS-3 at concentrations of 2 ppm to 5 ppm, were determined to be refreshing, comfortable and well tolerated by the patients.

A similar evaluation of compositions containing the cooling agent WS-23 at concentrations of 10 ppm to 20 ppm was also conducted. The compositions also produced a cooling effect and were comfortable.

1. An ophthalmic composition containing a cooling effective amount of a cooling agent selected from the group consisting of menthyl esters, carboxamides, menthane glycerol ketals, alkyl substituted ureas, sulfonamides, terpene analogs, furanones, phosphine oxides, and combinations thereof; and an ophthalmically acceptable vehicle therefor.

2. A composition according to claim 1, wherein the concentration of the cooling agent is 1 to 50 ppm.

3. A composition according to claim 2, wherein the cooling agent is selected from the group consisting of N,2,3-Trimethyl-2-isopropylbutamide and N-ethyl-5-methyl-2-(1-methylethyl), and combinations thereof.

4. A composition according to claim 1, wherein the vehicle comprises an aqueous solution that forms a gel or partial gel upon application to the eye.

5. A composition according to claim 4, wherein the aqueous solution contains a galactomannan polymer.

6. A composition according to claim 5, wherein the galactomannan polymer comprises guar or a derivative thereof.
